(19) United States
(12) Patent Application Publication
(10) Pub. No.: US 2021/0406738 A1
(43) Pub. Date: Dec. 30, 2021

(54) METHODS AND SYSTEMS FOR PROVIDING ACTIVITY FEEDBACK UTILIZING COGNITIVE ANALYSIS

(71) Applicant: INTERNATIONAL BUSINESS MACHINES CORPORATION, Armonk, NY (US)

(72) Inventors: Fearghal O'DONNCHA, Aran Islands (IE); Emanuele RAGNOLI, Mulhuddart (IE); Marco Luca SBODIO, Castaheany (IE)

(73) Assignee: INTERNATIONAL BUSINESS MACHINES CORPORATION, Armonk, NY (US)

(21) Appl. No.: 16/946,599

(22) Filed: Jun. 29, 2020

(51) Int. Cl. G06N 5/04; G06F 40/40

(52) U.S. Cl. CPC G06N 5/043; H04L 67/10; G06F 40/40

(57) ABSTRACT

Embodiments for providing activity feedback are provided. Information associated with a user performing an activity is received. A user biomechanical representation is generated based on the received information. A corpus associated with the activity is analyzed. An ideal biomechanical representation is generated based on the analyzing of the corpus associated with the activity. The user biomechanical representation is compared to the ideal biomechanical representation. Feedback for the user is generated based on the comparison of the user biomechanical model to the ideal biomechanical representation.

### BACKGROUND OF THE INVENTION

### Field of the Invention

The present invention relates in general to computing systems, and more particularly, to various embodiments for providing activity feedback to users utilizing cognitive analysis.

### Description of the Related Art

As users' connectivity to computing devices (e.g., mobile electronic devices, wearable devices, vehicular computing systems, etc.) increases, there is an ever-growing opportunity for the users to utilize and/or interact with the devices when performing various activities. For example, in recent years, various types of mobile electronic devices (e.g., mobile phones, wearable devices, etc.) and applications have been tailored for use in exercise and fitness related activities.

Many such systems provide the general ability to monitor exercise activity and various biometric data (e.g., heart rate, breathing patterns, etc.) and may give the user general feedback. However, the ability of even the most advanced systems currently available to compare the user's performance to a benchmark and provide detailed feedback to assist the user in making corrections is very limited.

### SUMMARY OF THE INVENTION

Various embodiments for providing activity feedback, by a processor, are provided. Information associated with a user performing an activity is received. A user biomechanical representation is generated based on the received information. A corpus associated with the activity is analyzed. An ideal biomechanical representation is generated based on the analyzing of the corpus associated with the activity. The user biomechanical representation is compared to the ideal biomechanical representation. Feedback for the user is generated based on the comparison of the user biomechanical model to the ideal biomechanical representation.

In addition to the foregoing exemplary embodiment, various other system and computer program product embodiments are provided and supply related advantages. The foregoing Summary has been provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a block diagram depicting an exemplary computing node according to an embodiment of the present invention;

FIG. 2 is an additional block diagram depicting an exemplary cloud computing environment according to an embodiment of the present invention;

FIG. 3 is an additional block diagram depicting abstraction model layers according to an embodiment of the present invention;

FIG. 4 is a block diagram of a method for providing activity feedback according to an embodiment of the present invention;

FIG. 5 is a schematic view of an environment in which the methods and systems described herein may be utilized according to an embodiment of the present invention; and

FIG. 6 is a flowchart diagram of an exemplary method for providing activity feedback according to an embodiment of the present invention.

### DETAILED DESCRIPTION OF THE DRAWINGS

As discussed above, as users' connectivity to computing devices (e.g., mobile electronic devices, wearable devices, vehicular computing systems, etc.) increases, there is an ever-growing opportunity for the users to utilize and/or interact with the devices when performing various activities. For example, in recent years, various types of mobile electronic devices (e.g., mobile phones, wearable devices, etc.) and applications have been tailored for use in exercise and fitness related activities.

Many such systems provide the general ability to monitor exercise activity and various biometric data (e.g., heart rate, breathing patterns, etc.) and may give the user general feedback. However, the ability of even the most advanced systems currently available to compare the user's performance to a benchmark and provide detailed feedback to assist the user in making corrections is very limited.

Given the amount of amount of data that modern computing systems are able to collect, along with ever-increasing performance of mobile devices, this development has the potential to significantly impact, if not disrupt or even at least partially replace, the rather significant person training industry. This industry now accounts for billions of dollars of revenue each year and is continuing to grow.

Current implementations of the utilization of such devices and applications for exercise/fitness provide limited functionality. For example, some current systems provide a “physical avatar” that guides personal movement when undertaking exercises. The physical avatar is simply a set of fixed points based on the position of the user's head, shoulders, knees, toes, etc. Some systems focus on monitoring the velocity of a particular piece of exercise equipment as it relates to a particular training methodology, while others require a physical sensor to be connected to the user's body.

To address these needs and/or the shortcomings in the prior art, in some embodiments described herein, methods and/or systems are disclosed that, for example, utilize capturing the user (e.g., via computer vision techniques) and an artificial intelligence (AI) (or cognitive analysis, etc.) system to monitor (or detect data) while a user performs an activity (e.g., exercise, physical activity, fitness activity, gym activity, etc.), assess the deviation between the user's performance and “correct” (or “ideal”) performance (or biometric performance), and provide the user with feedback to assist them in making corrections (e.g., in a manner similar to that of a human personal trainer).

In some embodiments, the system evaluates the degree, nature, and severity of deviation and recommends corrective feedback. The corrective feedback (and/or the analyzing of the user's performance in general) may be based on a knowledge base (e.g., a corpus) related to the activity (e.g., personal training, exercise, physical therapy, etc.), such as various types of documents, literature, etc. (e.g., related to exercise, physical therapy, medicine, etc.). The corrective feedback may be in the form of, for example, basic, corrective guidance, or in the case of severe deviation, a program (e.g., an exercise program) tailored to assist the user of a relatively long period of time (e.g., to help the user improve “core” strength, flexibility, or other biomechanical deviation/issue).

In some embodiments, the user (or users) is monitored or captured using a sensor to detect/collect information about the user's performance (or biomechanics) while performing the activity (e.g., an exercise). For example, a camera (or camera system) with visual recognition capabilities (e.g., computer vision) may be utilized. The visual recognition functionality may be implemented locally (e.g., on an edge device) or remotely (e.g., on the “cloud,” Internet, etc.). The visual recognition functionality may be utilized to assess exercise performance and extract key information (e.g. position of feet hip and shoulders, movement velocity, etc.) related to movement biomechanics compared to correct/ideal biomechanics for the particular exercise being performed (or a range of different exercises).

In some embodiments, the system generates (or utilizes) a biomechanical model(s) that is utilized to generate a representation (e.g., mathematical representation) of the user's performance and a representation of the correct performance. Such may be performed utilizing various biometric data about the user (e.g., height, weight, body mass index (BMI), etc.).

An AI system may be utilized to assess movement biomechanics of the user compared to correct biomechanics for the exercise (e.g., the range of movement). In some embodiments, the AI system is trained on (and/or analyzes) a corpus (e.g., one or more documents) associated with the exercise, physical therapy, etc. Such is utilized to evaluate the deviation(s) in the user's performance of the exercise from correct/ideal biomechanics. The system may also generate one or more recommendation for corrective actions and provide such to the user (e.g., via visual and/or aural indications rendered via any suitable device).

Additionally, the system may have the ability to classify the severity of the deviation. If the deviation is minor (e.g., below a first threshold), the user may be provided with positive feedback (i.e., a first type of feedback). If the deviation is more significant (e.g., between the first threshold and a second threshold), the feedback may include suggested corrective actions (e.g., adjusting the distance between feet, standing more upright, etc.) that may be implemented and immediately beneficial to the user. If the deviation is severe (e.g., above the second threshold), the feedback may include a more complex set of recommendations which may include a particular exercise program to correct biomechanics over time (which the system may determine), such as an exercise program to be utilized by the user over the course of weeks, months, etc.

As such, the system may (e.g., utilizing computing vision techniques and AI) evaluate the “correctness” of the user's performance of an exercise(s) and provide corrective recommendations if required (e.g., according to user-defined constraints, such as the number of days per week the exercise is done, etc.).

The methods and systems described herein may provide users with a “digital personal trainer,” which reduces potential of injury, improves athletic performance, and avoids the costs of a (human) personal trainer. Additionally, the methods and systems allow administrators to tap into a large, lucrative industry with ever-increasing demand, while also combating society's rising health-related concerns.

It should be understood that at least some of the aspects of functionality described herein may be performed utilizing a cognitive analysis (or AI, machine learning (ML), etc.). The cognitive analysis may include natural language processing (NLP) and/or natural language understanding (NLU) or NLP/NLU technique, such classifying natural language, analyzing tone, and analyzing sentiment (e.g., scanning for keywords, key phrases, etc.) with respect to, for example, content (e.g., of a corpus) and communications sent to and/or received by users or entities and/or other available data sources. In some embodiments, Mel-frequency cepstral coefficients (MFCCs) (e.g., for audio content), and/or region-based convolutional neural network (R-CNN) pixel mapping (e.g., for object detection/classification and facial recognition in images/videos), as are commonly understood, are used.

The processes described herein may utilize various information or data sources associated with users, entities and/or the content of documents. The data sources may include any available information (or data) sources. For example, in some embodiments, a profile (e.g., a cognitive profile) for the user(s) (and/or entities) may be generated. Data sources that may be use used to generate cognitive profiles may include any appropriate data sources associated with the user/entity that are accessible by the system (perhaps with the permission or authorization of the user/entity). Examples of such data sources include, but are not limited to, communication sessions and/or the content (or communications) thereof (e.g., phone calls, video calls, text messaging, emails, in person/face-to-face conversations, etc.), a profile of (or basic information about) the user/entity (e.g., demographic information, job title, place of work, length of time at current position, family role, etc.), a schedule or calendar (i.e., the items listed thereon, time frames, etc.), projects (e.g., past, current, or future work-related projects, “to-do” lists, etc.), location (e.g., previous and/or current location and/or location relative to other users), social media activity (e.g., posts, reactions, comments, groups, etc.), browsing history (e.g., web pages visited), and online purchases. The cognitive profile(s) may be utilized to, for example, tailor the feedback to the individual user(s).

As such, in some embodiments, the methods and/or systems described herein may utilize a “cognitive analysis,” “cognitive system,” “machine learning,” “cognitive modeling,” “predictive analytics,” and/or “data analytics,” as is commonly understood by one skilled in the art. Generally, these processes may include, for example, receiving and/or retrieving multiple sets of inputs, and the associated outputs, of one or more systems and processing the data (e.g., using a computing system and/or processor) to generate or extract models, rules, etc. that correspond to, govern, and/or estimate the operation of the system(s), or with respect to the embodiments described herein, providing activity feedback, as described herein. Utilizing the models, the performance (or operation) of the system (e.g., utilizing/based on new inputs) may be predicted and/or the performance of the system may be optimized by investigating how changes in the input(s) effect the output(s). Feedback received from (or provided by) users and/or administrators may also be utilized, which may allow for the performance of the system to further improve with continued use.

It should be understood that as used herein, the term “computing node” (or simply “node”) may refer to a computing device, such as a mobile electronic device, desktop computer, etc. and/or an application, such a chatbot, an email application, a social media application, a web browser, etc. In other words, as used herein, examples of computing nodes include, for example, computing devices such as mobile phones, tablet devices, desktop computers, or other devices, such as appliances (IoT appliances) that are owned and/or otherwise associated with individuals (or users), and/or various applications that are utilized by the individuals on such computing devices.

In particular, in some embodiments, a method for providing activity feedback, by a processor, is provided. Information associated with a user performing an activity is received. A user biomechanical representation is generated based on the received information. A corpus associated with the activity is analyzed. An ideal biomechanical representation is generated based on the analyzing of the corpus associated with the activity. The user biomechanical representation is compared to the ideal biomechanical representation. Feedback for the user is generated based on the comparison of the user biomechanical model to the ideal biomechanical representation.

The received information may be detected utilizing a camera. At least one of the generating of the user biomechanical representation and the generating of the ideal biomechanical representation may be performed utilizing a cognitive analysis. The analyzing of the corpus associated with the activity may be performed utilizing natural language processing.

If a difference between the user biomechanical representation and the ideal biomechanical representation is less than a first threshold, a first type of feedback (e.g., positive feedback) may be provided to the user. If the difference between the user biomechanical representation and the ideal biomechanical representation is between the first threshold and a second threshold, a second type of feedback (e.g., relatively simple correctional feedback) may be provided to the user. If the difference between the user biomechanical representation and the ideal biomechanical representation is greater than the second threshold, a third type of feedback (e.g., relatively complex/involved feedback) may be provided to the user.

Physical metrics associated with the user may be received. At least one of the generating of the user biomechanical representation and the generating of the ideal biomechanical representation may be based on the received physical metrics. An indication of the generated feedback may be caused to be provided to the user. The indication may include at least one of a visual indication and an aural indication.

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment, such as cellular networks, now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

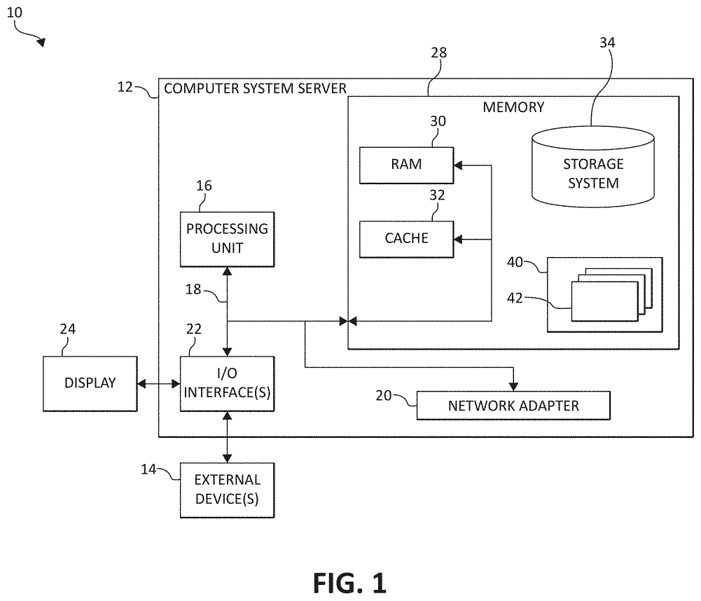

Referring now to FIG. 1, a schematic of an example of a cloud computing node is shown. Cloud computing node 10 is only one example of a suitable cloud computing node and is not intended to suggest any limitation as to the scope of use or functionality of embodiments of the invention described herein. Regardless, cloud computing node 10 (and/or one or more processors described herein) is capable of being implemented and/or performing (or causing or enabling) any of the functionality set forth hereinabove.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 1, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, system memory 28 may include at least one program product having a set (e.g., at least one) of program modules that are configured to carry out the functions of embodiments of the invention.

Program/utility 40, having a set (at least one) of program modules 42, may be stored in system memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples include, but are not limited to: microcode, device drivers, redundant processing units, external disk drive arrays, RAID systems, tape drives, and data archival storage systems, etc.

In the context of the present invention, and as one of skill in the art will appreciate, various components depicted in FIG. 1 may be located in, for example, personal computer systems, server computer systems, thin clients, thick clients, hand-held or laptop devices, multiprocessor systems, microprocessor-based systems, set top boxes, programmable consumer electronics, network PCs, mobile electronic devices such as mobile (or cellular and/or smart) phones, personal data assistants (PDAs), tablets, wearable technology devices, laptops, handheld game consoles, portable media players, etc., as well as computing systems in (and/or integrated into) vehicles, such as automobiles, aircraft, watercrafts, etc. However, in some embodiments, some of the components depicted in FIG. 1 may be located in a computing device in, for example, a satellite, such as a Global Position System (GPS) satellite. For example, some of the processing and data storage capabilities associated with mechanisms of the illustrated embodiments may take place locally via local processing components, while the same components are connected via a network to remotely located, distributed computing data processing and storage components to accomplish various purposes of the present invention. Again, as will be appreciated by one of ordinary skill in the art, the present illustration is intended to convey only a subset of what may be an entire connected network of distributed computing components that accomplish various inventive aspects collectively.

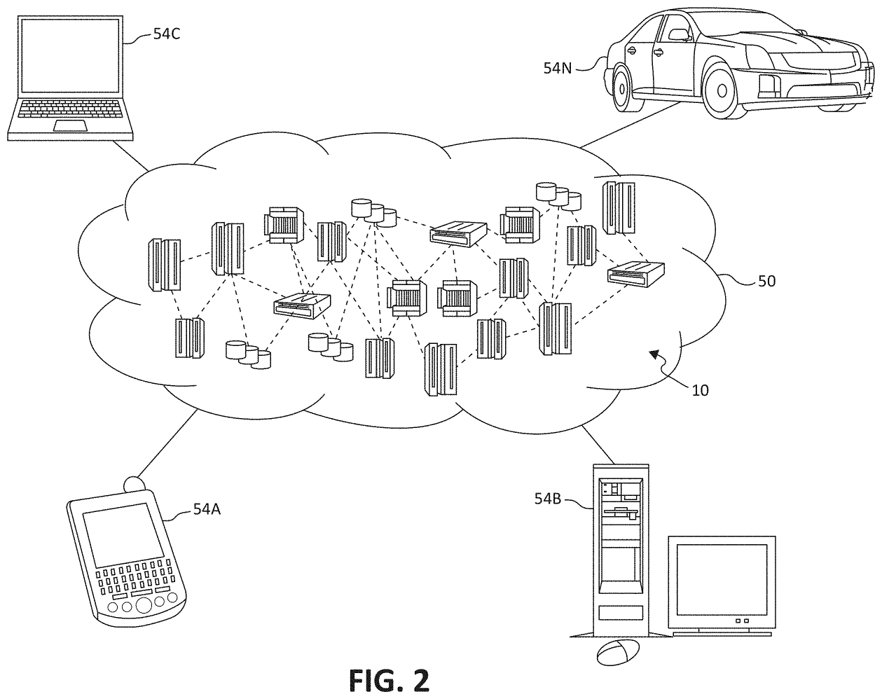

Referring now to FIG. 2, illustrative cloud computing environment 50 is depicted. As shown, cloud computing environment 50 comprises one or more cloud computing nodes 10 with which local computing devices used by cloud consumers, such as, for example, cellular (or mobile) telephone or PDA 54A, desktop computer 54B, laptop computer 54C, and vehicular computing system (e.g., integrated within automobiles, aircraft, watercraft, etc.) 54N may communicate.

Still referring to FIG. 2, nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 2 are intended to be illustrative only and that computing nodes 10 and cloud computing environment 50 can communicate with any type of computerized device over any type of network and/or network addressable connection (e.g., using a web browser).

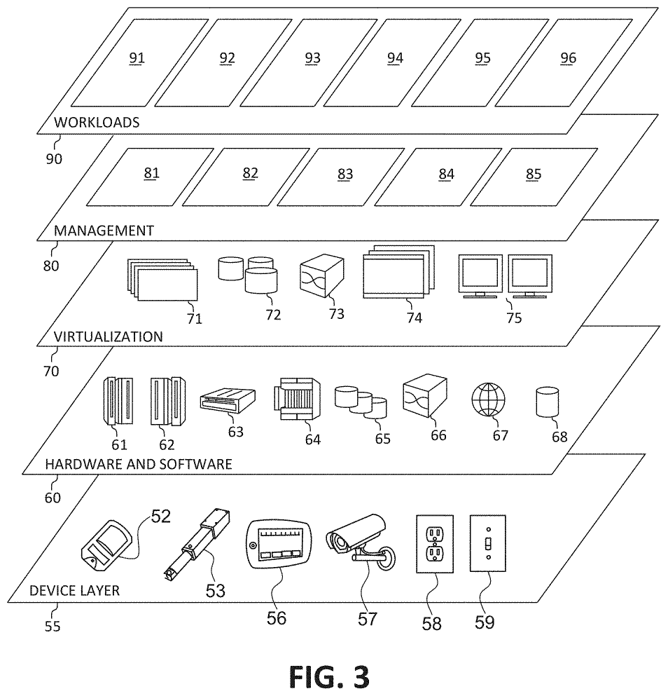

Referring now to FIG. 3, a set of functional abstraction layers provided by cloud computing environment 50 (FIG. 2) is shown. It should be understood in advance that the components, layers, and functions shown in FIG. 3 are intended to be illustrative only and embodiments of the invention are not limited thereto. As depicted, the following layers and corresponding functions are provided:

Device layer 55 includes physical and/or virtual devices, embedded with and/or standalone electronics, sensors, actuators, and other objects to perform various tasks in a cloud computing environment 50. Each of the devices in the device layer 55 incorporates networking capability to other functional abstraction layers such that information obtained from the devices may be provided thereto, and/or information from the other abstraction layers may be provided to the devices. In one embodiment, the various devices inclusive of the device layer 55 may incorporate a network of entities collectively known as the “internet of things” (IoT). Such a network of entities allows for intercommunication, collection, and dissemination of data to accomplish a great variety of purposes, as one of ordinary skill in the art will appreciate.

Device layer 55 as shown includes sensor 52, actuator 53, “learning” thermostat 56 with integrated processing, sensor, and networking electronics, camera 57, controllable household outlet/receptacle 58, and controllable electrical switch 59 as shown. Other possible devices may include, but are not limited to, various additional sensor devices, networking devices, electronics devices (such as a remote control device), additional actuator devices, so called “smart” appliances such as a refrigerator, washer/dryer, or air conditioning unit, and a wide variety of other possible interconnected devices/objects.

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provides cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provides pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and, in the context of the illustrated embodiments of the present invention, various workloads and functions 96 for providing activity feedback, as described herein. One of ordinary skill in the art will appreciate that the workloads and functions 96 may also work in conjunction with other portions of the various abstractions layers, such as those in hardware and software 60, virtualization 70, management 80, and other workloads 90 (such as data analytics processing 94, for example) to accomplish the various purposes of the illustrated embodiments of the present invention.

As previously mentioned, in some embodiments, methods and/or systems are provided that, for example, utilize capturing the user (e.g., via computer vision techniques) and an artificial intelligence (AI) (or cognitive analysis, etc.) system to monitor (or detect data) while a user performs an activity (e.g., exercise, fitness activity, gym activity, etc.), assess the deviation between the user's performance and “correct” (or “ideal”) performance (or biometric performance), and provide the user with feedback to assist them in making corrections (e.g., in a manner similar to that of a human personal trainer).

That is, in some embodiments, the methods/systems monitor exercise performance (e.g., bench pressing, etc.), evaluates the user's biomechanics, and provides actionable feedback based on correct biomechanics. The systems may provide (and/or utilize) a cognitive system to inform the user of incorrect biomechanics during exercise performance and provide feedback in terms of recommended corrections (e.g., position of feet and hips, head position, angle of back or shins, etc.) to align with correct biomechanics. The system may evaluate the degree or severity of deviation from correct biomechanics. In some situations, the system may generate (or determine, compose, etc.) a program for the user to correct deviations based on an analysis of a corpus of exercise/physical therapy literature (e.g., a program to improve core strength, increase ankle mobility, increase muscular flexibility, etc.).

In some embodiments, the system utilizes the ability to monitor activity performance using computer vision and extract user biomechanics (e.g., foot/hip/shoulder position, angle of limbs and trunk, etc.). The system may also utilize (and/or generate) a biomechanical model that constructs a virtual representation of user biomechanics based on body measures and model parameters. A machine learning (ML) (or AI, cognitive analysis, etc.) component may be utilized to extract biomechanical model parameters from user biomechanics and a corpus of model parameters (i.e., from the analyzed corpus). An NLP module may be utilized to analyze a (or the) corpus of annotated literature from the physical therapy/exercise domain and relate classified biomechanics with appropriate diagnoses and physical treatment.

A user interface (UI) (e.g., rendered by a display device) and/or conversation component (e.g., a speaker and/or microphone) may be utilized to provide the feedback, generated based on the biomechanical analysis, to the user. The feedback may include relatively simple “pointers” in cases of minor deviations (e.g., “position feet wider apart” or “keep head in upright position”). However, in cases of more significant deviation, an exercise or activity program may be generated and provided (e.g., a core strengthening program if the user is leaning too far forward during a particular exercise or an ankle mobility program if the user is exhibiting incorrect knee position).

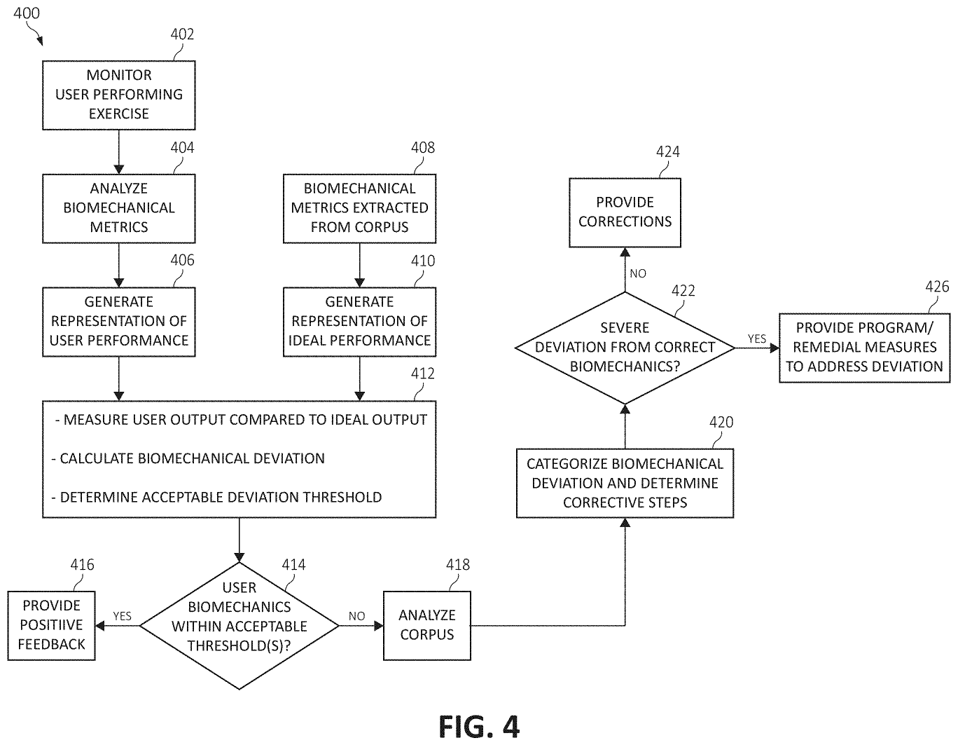

FIG. 4 illustrates a block diagram of a method (and/or system) 400 for providing activity (e.g., exercise) feedback according to an embodiment of the present invention. The method 400 may utilize various body measures (or biometric attributes), a biomechanical model, a corpus of biomechanical model inputs/parameters or trained ML model, and a corpus of associated content as input, as described in greater detail below.

At block 402, a user performing an exercise (or other activity) is monitored (or captured, recorded, etc.) with a sensor or recording device, such as a camera. In some embodiments, a computer vision technique is utilized to extract imagery from the captured information at particular intervals (e.g., once per second, every five seconds, etc.) while the exercise is being performed. At block 404, the biomechanics (or biomechanical metrics or parameters) of the user (as appearing in the captured information) are analyzed (e.g., utilizing ML). At block 406, biomechanical parameters are fed into the biomechanical model (or ensemble of biomechanical models) to generate a numerical (or mathematical) representation of the user's performance, which is provided to block 412.

At block 408, the biometric parameters of the user are provided and information related to the correct/ideal performance of the exercise is extracted from the corpus (and/or a database). The biometric parameters may be manually provided by the user (e.g., input via a computing device), perhaps along with a description or selection of the exercise (or other activity) being performed. However, in some embodiments, the system may automatically determine the type of exercise (or other activity) being performed (e.g., via object detection, visual recognition, etc. performed on received images).

At block 410, the biometric parameters of the user are utilized together with the information extracted from the corpus (perhaps along with the biomechanical model) to generate a numerical representation of the correct/ideal performance of the exercise. Additionally, thresholds for acceptable deviation from this representation may also be determined. The output of block 408 is provided to block 412.

At block 412, several processes may be performed. The user output (or performance) may be measured against (or compared to) the correct output (or performance). Additionally, the biomechanical deviation (of difference) between the user performance and the correct performance may be calculated (or determined, computed, etc.). The deviation may be determined as a numerical value (e.g., percentage, integer, etc.) or a “grade” (e.g., “low,” “high,” etc.) Further, the acceptable deviation(s) or threshold(s) to be utilized for the user may be determined or selected (e.g., based on the user's age, BMI, etc.).

Still referring to FIG. 4, at block 414, it is determined whether or not the user's biomechanics are within the acceptable threshold(s). More particularly, it may be determined if the deviation from the correct performance is below (or within) a first threshold (or set of thresholds). If so, at block 416 positive feedback (e.g., “good job,” etc.) is generated and provided to the user (i.e., because the user's performance is relatively similar to the correct/ideal performance and/or the user is performing the exercise relatively correctly). Such feedback may be provided (or generated) in any suitable manner, such as those described below.

If the user's biomechanics are not within the acceptable threshold(s), at block 418, the corpus (e.g., related to the exercise, medicine, physical therapy, etc.) is analyzed (e.g., via NLP/NLU) to extract information related to biomechanics (e.g., classified biomechanics). It should be noted that this process may (at least partially) be performed before the method 400 is initiated (e.g., before the user begins the activity/exercise).

At block 420, the biomechanical deviation is (further) categorized (and/or analyzed) and corrective steps are extracted from the corpus. At block 422, it is determined whether or not the deviation(s) from the correct biomechanics is severe. If the deviation is not severe (e.g., the deviation is between the first threshold and a second threshold), at block 424, relatively simple, immediate corrective feedback is generated and provided to the user (e.g., related to foot position, back alignment, etc.). This feedback may be provided utilizing a display device and/or conversational assistant (e.g., visual indications and/or aural indications) implemented through a suitable computing device (e.g., a mobile electronic device, a computing system integrated into activity/exercise equipment, etc.).

If the deviation is severe (e.g., the deviation is above the second threshold), at block 426, the feedback provided may (also) include a recommended exercise program or remedial measures to address a biomechanical issue(s) (e.g., core strengthening, mobility, medical consultation, etc.). This feedback may be provided in any suitable manner (e.g., electronic communication, such as email, text message, etc., aural indications, etc.).

As alluded to above, the methods and system described herein may utilize various body measures (or biometric attributes) of the user(s) to perform the functionality described herein. These measures may include, for example, height, weight, BMI, age, and any other pertinent measure that is associated with biomechanical modeling an analysis. In some embodiments, the recording (or capturing) device utilized is a camera (or camera system with computer vision capabilities), which may be utilized to continuously record the user performing the activity. For example, a camera may be attached to exercise equipment or integrated into the user's mobile electronic device (e.g., a mobile phone or tablet). The camera may be aligned in a particular manner (e.g., in a particular recording position) and produce an output of a set of parameters used as inputs for the biomechanical model (e.g. trunk flexion, hip flexion/extension, knee flexion/extension, etc.) and/or any inputs pertinent to biomechanical analysis. The computer vision technique utilized may identify joint centers and/or body landmarks used to define rigid segments representing, for example, a component of exercise equipment, the user's trunk, the user's thighs, the user's feet, etc.

The biomechanical model(s) may include a virtual human model and/or simulation software used to construct a numerical representation of activity performance based on body measures and outputs from the computer vision. Classified biomechanics may refer to a corpus of biomechanical model inputs of ML models trained based on a corpus or a database of model parameters and associated body measures used to extract inputs or parameters for a biomechanical model. The corpus (and/or documents) utilized may include any documents (e.g., scholarly papers, articles, books, etc.), web pages, etc. related to exercise, physical therapy, medicine, or any other field that may be pertinent to providing feedback to a user performing an exercise or other activity. The corpus may be annotated and be utilized to train the biomechanical model (e.g., as related to the user's performance and/or the correct performance).

The output of the computer vision component may be provided as input to a ML model that extracts biomechanical model parameters from measures of biomechanical metrics and the information on body measures (e.g. height, weight, etc.). A related set of model parameters may be extracted based on inputs of body measures (e.g., heights, weight, etc.) and a correct or representative set of measures for activity (e.g., exercise) performance (e.g., correct trunk flexion, hip flexion/extension, knee flexion and extension, and ankle dorsiflexion/plantar flexion, etc.).

In some embodiments, a biomechanical model of the user's (activity/exercise) performance is created (e.g. using virtual human modeling and simulation software) using as inputs model parameters extracted from the machine learning analysis and user body measures (height, weight, etc.). Also, a biomechanical model of the correct performance may be created utilizing the same body measures and the model parameters extracted from the corpus (e.g., a reference/correct/ideal biomechanical model). Utilizing the reference model and body measurements of the user, a set of acceptable thresholds of deviation may be determined based on, for example, ensemble modeling simulations. As such, in some embodiments, the biomechanical model is utilized to generate a biomechanical model of the user, a reference biomechanical model (e.g., based on the user's biometric attributes), and thresholds for deviation from the reference model.

In some embodiments, an analytics (or cognitive analysis) module calculates the difference (or deviation) between the user biomechanical model and the reference model. Based on the difference and acceptable thresholds, it is determined whether or not performance is within acceptable thresholds. Such may be provided to a communication module, along with information related to the degree of deviation from correct performance.

As described above, a corpus of annotated physical therapy literature may be analyzed to extract information pertinent to specific deviation (e.g., of trunk flexion (relative to the vertical axis), hip flexion/extension, knee flexion and extension, etc.). Identified deviation (and degree of deviation) from acceptable deviation may be associated with a recommended treatment program from physical therapy using a semantic matching module based on a defined ontology. For example, a lexical matching may be conducted to directly connect observed biomechanical deviation with that from the literature (e.g., ‘hip flexion too short,” “vertical position of trunk too low,” etc.) and relate such to recommended treatment (e.g., mobility program or core strengthening exercise). Then, a second semantic matching module may relate biomechanical deviation and recommended treatment accounting for different conventions in the literature.

In this manner, the methods and systems described herein may provide feedback in the form of recommended activity/exercise programs or remediation processes. That is, if the user is determined to be utilizing incorrect biomechanics with a minor deviation from the correct biomechanics, they may be provided with feedback that includes corrections, such as foot position, etc. However, if the deviation is major, the user may be informed of such and the recommendation may include more in-depth, time-consuming changes, such as new exercise programs, seeking medical advice, etc. that may be carried out over a significant period of time (e.g., weeks, months, etc.).

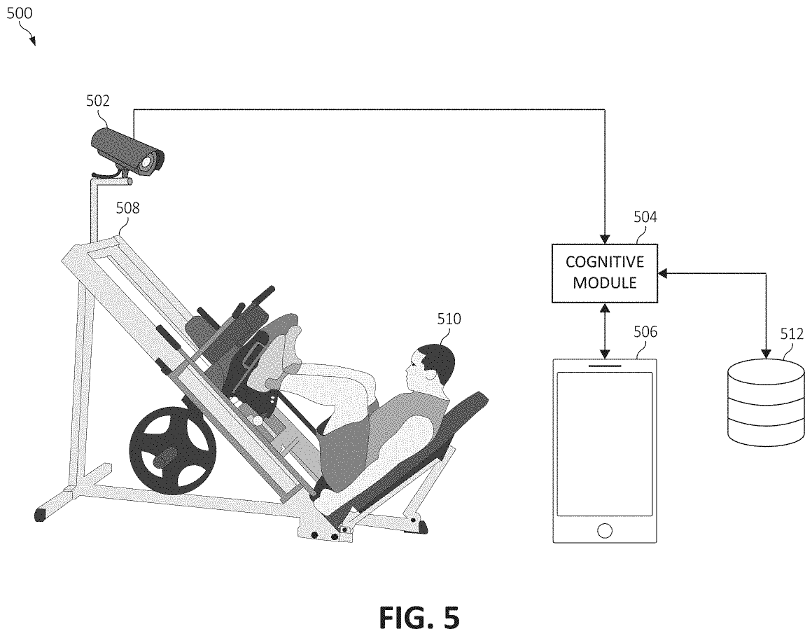

Referring now to FIG. 5, an environment 500 in which the methods and systems described herein may be utilized is shown. The environment 500 includes a sensor 502, a cognitive module 504, a computing device 506, a piece of exercise equipment 508 (i.e., being utilized by a user 510), and a database 512.

In some embodiments, the sensor 502 includes a camera, which in the depicted embodiment, is attached to the exercise equipment 508. However, it should be understood that in other embodiments, the sensor may be connected to and/or integrated into a different device (e.g., a mobile electronic device) or a “stand alone” camera (e.g., simply set on the ground, attached to a wall, etc.). In particular, the camera may be arranged such that is may capture/record the user (or the user's body) as they perform an exercise associated with the exercise equipment 508. In the particular example shown, the exercise is a horizontal leg press. However, it should be understood that is merely intended as an example, as the methods/system described herein may be applied to any type of activity or exercise (even those that don't require specific equipment, such as push ups, running, etc.).

The cognitive module 504 may include any suitable computing device that is configured to perform at least some of the processes, functionality, etc. described herein, including ML techniques, cognitive analyzes, NLP/NLU, etc. The computing device 506 may be any suitable computing device through which the user 510 may interact with the system (e.g., receive feedback, etc.) and may include a display screen, one or more speakers, and a microphone. Although the computing device 506 shown in a mobile phone, it should be understood that other types of computing devices may be utilized, such as other mobile electronic devices (e.g., wearable devices) and computing devices integrated with the exercise equipment. The database 512 may include (or have stored thereon) any corpus (e.g., one or more documents) related to exercise, physical therapy, medicine, etc., including information sources available through online channels (e.g., web pages, etc.).

It should be understood that at least some of the components shown in FIG. 5 may be located remotely and in operable communication via any suitable communications network. For example, information captured by the camera may be sent to the cognitive module 504 via the cloud, which accesses corpora (i.e., the database 512) through online channels. Output generated by the cognitive module may then be sent to the computing device 506 (e.g., located near the user 510 and exercise equipment 508) to be viewed by the user 510. However, in some embodiments, at least some of the functionality performed by the cognitive module 504 may be integrated into and/or performed by the computing device 506.

In some embodiments, methods and/or systems for monitoring exercise performance (e.g., a gym squat), evaluating user biomechanics, and providing actionable feedback based on correct biomechanics are provided. A cognitive system may be utilized to inform the user of incorrect biomechanics during exercise performance and provide feedback in terms of recommended corrections (e.g., position of feet and hips, head position, angle of back or shins, etc.) to align with correct biomechanics. The systems may evaluate the degree or severity of deviation from correct biomechanics and provide an exercise program to correct the deviations based on an analysis of a corpus of physical therapy literature (e.g. a program to improve core strength, increase ankle mobility or muscular flexibility, etc).

The systems may provide the ability to monitor activity or exercise performance using computer vision and extract user biomechanics. A biomechanical model that constructs virtual representation of user biomechanics based on body measures and model parameters may be utilized (or provided). A machine learning component that extracts biomechanical model parameters from user biomechanics and corpus of model parameters may be utilized. An NLP module that analyzes a corpus of annotated literature from the physical therapy domain and relates classified biomechanics with appropriate diagnoses and physical treatment may be utilized. A UI or conversation component that provides feedback to the user based on biomechanics analysis and/or receives feedback from the user in terms of user rating, medical diagnosis, etc. may be provided.

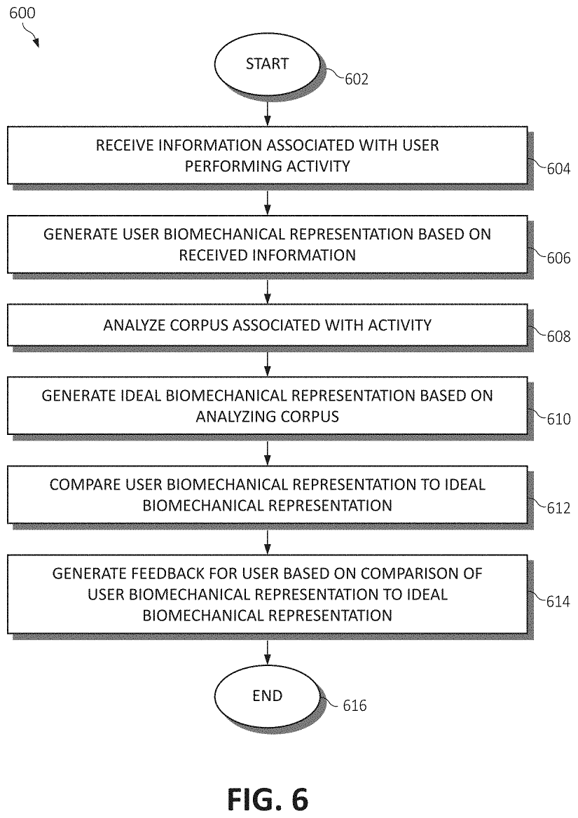

Turning to FIG. 6, a flowchart diagram of an exemplary method 600 for providing activity (e.g., physical activity, exercise, etc.) feedback is provided. The method 600 begins (step 602) with, for example, a user (or individual) performing an activity and/or a system configured with the functionality described being deployed near activity or exercise equipment (e.g., in a gym).

Information associated with a user performing the activity (e.g., exercise) is received (step 604). The information may be detected or captured with a camera (e.g., recording/capturing a user as they perform an exercise).

A user biomechanical representation is generated based on the received information (step 606). Physical metrics associated with the user may (also) be received, which may be utilized to generate the user biomechanical representation.

A corpus associated with the activity is analyzed (step 608). The corpus may include one or more document, web page, etc. related to the activity, exercise, physical therapy, medicine, etc. (e.g., including those available through online channels). The analyzing of the corpus associated with the activity may be performed utilizing natural language processing.

An ideal (or “correct”) biomechanical representation is generated based on the analyzing of the corpus associated with the activity (step 610). At least one of the generating of the user biomechanical representation and the generating of the ideal biomechanical representation may be performed utilizing a cognitive analysis.

The user biomechanical representation is compared to the ideal biomechanical representation (step 612). At least one of the generating of the user biomechanical representation and the generating of the ideal biomechanical representation may be based on the received physical metrics.

Feedback for the user is generated based on the comparison of the user biomechanical model to the ideal biomechanical representation (step 614). If a difference between the user biomechanical representation and the ideal biomechanical representation is less than a first threshold, a first type of feedback (e.g., positive feedback) may be provided to the user. If the difference between the user biomechanical representation and the ideal biomechanical representation is between the first threshold and a second threshold, a second type of feedback (e.g., relatively simple correctional feedback) may be provided to the user. If the difference between the user biomechanical representation and the ideal biomechanical representation is greater than the second threshold, a third type of feedback (e.g., relatively complex/involved feedback) may be provided to the user. An indication of the generated feedback may be caused to be provided to the user. The indication may include at least one of a visual indication and an aural indication.

Method 600 ends (step 616) with, for example, any correctional feedback provided being utilized by the user as they continue to perform the activity. In some embodiments, feedback from users may also be utilized to improve the performance of the system over time.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowcharts and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowcharts and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowcharts and/or block diagram block or blocks.

The flowcharts and block diagrams in the figures illustrate the architecture, functionality, and operation of possible implementations of systems, methods, and computer program products according to various embodiments of the present invention. In this regard, each block in the flowcharts or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts or carry out combinations of special purpose hardware and computer instructions.

1. A method for providing activity feedback, by a processor, comprising:
receiving information associated with a user performing an activity;
generating a user biomechanical representation based on said received information;
analyzing a corpus associated with the activity;
generating an ideal biomechanical representation based on the analyzing of the corpus associated with the activity;
comparing the user biomechanical representation to the ideal biomechanical representation; and
generating feedback for the user based on said comparison of the user biomechanical representation to the ideal biomechanical representation.

2. The method of claim 1, wherein said received information is detected utilizing a camera.

3. The method of claim 1, wherein at least one of the generating of the user biomechanical representation and the generating of the ideal biomechanical representation is performed utilizing a cognitive analysis.

4. The method of claim 1, wherein the analyzing of the corpus associated with the activity is performed utilizing natural language processing.

5. The method of claim 1, wherein if a difference between the user biomechanical representation and the ideal biomechanical representation is less than a first threshold, a first type of feedback is provided to the user, if the difference between the user biomechanical representation and the ideal biomechanical representation is between the first threshold and a second threshold, a second type of feedback is provided to the user, and if the difference between the user biomechanical representation and the ideal biomechanical representation is greater than the second threshold, a third type of feedback is provided to the user.

6. The method of claim 1, further comprising receiving physical metrics associated with the user, and wherein at least one of the generating of the user biomechanical representation and the generating of the ideal biomechanical representation is based on said received physical metrics.

7. The method of claim 1, further comprising causing an indication of said generated feedback to be provided to the user, wherein the indication includes at least one of a visual indication and an aural indication.

8. A system for providing activity feedback comprising:
a processor executing instructions stored in a memory device, wherein the processor:
receives information associated with a user performing an activity;
generates a user biomechanical representation based on said received information;
analyzes a corpus associated with the activity;
generates an ideal biomechanical representation based on the analyzing of the corpus associated with the activity;
compares the user biomechanical representation to the ideal biomechanical representation; and
generates feedback for the user based on said comparison of the user biomechanical model to the ideal biomechanical representation.

9. The system of claim 8, wherein said received information is detected utilizing a camera.

10. The system of claim 8, wherein at least one of the generating of the user biomechanical representation and the generating of the ideal biomechanical representation is performed utilizing a cognitive analysis.

11. The system of claim 8, wherein the analyzing of the corpus associated with the activity is performed utilizing natural language processing.

12. The system of claim 8, wherein if a difference between the user biomechanical representation and the ideal biomechanical representation is less than a first threshold, a first type of feedback is provided to the user, if the difference between the user biomechanical representation and the ideal biomechanical representation is between the first threshold and a second threshold, a second type of feedback is provided to the user, and if the difference between the user biomechanical representation and the ideal biomechanical representation is greater than the second threshold, a third type of feedback is provided to the user.

13. The system of claim 8, wherein the processor further receives physical metrics associated with the user, and wherein at least one of the generating of the user biomechanical representation and the generating of the ideal biomechanical representation is based on said received physical metrics.

14. The system of claim 8, wherein the processor further causes an indication of said generated feedback to be provided to the user, wherein the indication includes at least one of a visual indication and an aural indication.

15. A computer program product for providing activity feedback, by a processor, the computer program product embodied on a non-transitory computer-readable storage medium having computer-readable program code portions stored therein, the computer-readable program code portions comprising:
an executable portion that receives information associated with a user performing an activity;
an executable portion that generates a user biomechanical representation based on said received information;
an executable portion that analyzes a corpus associated with the activity;
an executable portion that generates an ideal biomechanical representation based on the analyzing of the corpus associated with the activity;
an executable portion that compares the user biomechanical representation to the ideal biomechanical representation; and
an executable portion that generates feedback for the user based on said comparison of the user biomechanical model to the ideal biomechanical representation.

16. The computer program product of claim 15, wherein said received information is detected utilizing a camera.

17. The computer program product of claim 15, wherein at least one of the generating of the user biomechanical representation and the generating of the ideal biomechanical representation is performed utilizing a cognitive analysis.

18. The computer program product of claim 15, wherein the analyzing of the corpus associated with the activity is performed utilizing natural language processing.

19. The computer program product of claim 15, wherein if a difference between the user biomechanical representation and the ideal biomechanical representation is less than a first threshold, a first type of feedback is provided to the user, if the difference between the user biomechanical representation and the ideal biomechanical representation is between the first threshold and a second threshold, a second type of feedback is provided to the user, and if the difference between the user biomechanical representation and the ideal biomechanical representation is greater than the second threshold, a third type of feedback is provided to the user.

20. The computer program product of claim 15, wherein the computer-readable program code portions further include an executable portion that receives physical metrics associated with the user, and wherein at least one of the generating of the user biomechanical representation and the generating of the ideal biomechanical representation is based on said received physical metrics.

21. The computer program product of claim 15, wherein the computer-readable program code portions further include an executable portion that causes an indication of said generated feedback to be provided to the user, wherein the indication includes at least one of a visual indication and an aural indication.
